(19) United States
(12) Patent Application Publication
(10) Pub. No.: US 2019/0030253 A1
(43) Pub. Date: Jan. 31, 2019

(54) ERGONOMIC SYRINGE

(71) Applicant: Synergize Co., Denver, CO (US)

(72) Inventor: Jennifer Barbour, Denver, CO (US)

(73) Assignee: Synergize Co., Denver, CO (US)

(21) Appl. No.: 16/147,514

(22) Filed: Sep. 28, 2018

Related U.S. Application Data
(63) Continuation-in-part of application No. 13/244,244, filed on Sep. 23, 2011, now abandoned.
Application No. 13/244,244 is a continuation-in-part of application No. 12/106,201, filed on Apr. 18, 2008, now abandoned.
(60) Provisional application No. 60/913,198, filed on Apr. 20, 2007.

(51) Int. Cl. A61M 5/31; A61M 5/315

(52) U.S. Cl. CPC A61M 5/3137; A61M 5/3135; A61M 5/3148; A61M 2205/586; A61M 2005/3126; A61M 2005/3118; A61M 2205/0216; A61M 5/31505

(57) ABSTRACT

The present invention relates to devices and methods for injection. More specifically, the devices and methods provide an ergonomic aspect surrounding syringes. In some embodiments, the devices and methods are especially useful in the administration of low volumes of dermal fillers to a subject.

### CROSS REFERENCE TO REFERENCE TO RELATED APPLICATIONS

This application claims priority as a continuation-in-part of U.S. patent application Ser. No. 13/244,244 filed Sep. 23, 2011 and entitled “ERGONOMIC SYRINGE,” which claims benefit of U.S. patent application Ser. No. 12/106,201 filed Apr. 18, 2008 and entitled “ERGONOMIC SYRINGE”, which claims priority to U.S. Provisional Application No. 60/913,198, filed Apr. 20, 2007. Each of these applications is hereby incorporated by reference in its entirety.

### FIELD OF THE INVENTION

A device and method relating to injection devices and methods are disclosed in accordance with preferred embodiments of the present invention. In some embodiments, the device and method provide for ease of use of injection devices. Certain embodiments surround the use of a device or method of certain embodiments to support increased dexterity and maintaining the sterile nature of a syringe and parts of a syringe.

### BACKGROUND OF THE INVENTION

Devices for delivery or injection of substances, such as syringes, are well-known in the art. Despite the importance of such devices and the fact that such devices are being used with ever increasing frequency, the art has seen little in the way of developments in regard to certain aspects of these injection devices.

### SUMMARY OF THE INVENTION

While much emphasis is placed on the comfort of a subject that is to receive an injection via a syringe, frequently, the comfort and/or convenience afforded the person using the syringe is ignored. Common syringes used for administering injections are configured for momentary use, and for administering an injection. As such, common syringes available in the art are limited in functionality to injecting a substance and not readily able to allow aspiration with a single hand. As a result, the person using the syringe is relegated to using two hands in order to aspirate the syringe. It will be appreciated by those skilled in the art, that aspirating a syringe involves drawing the plunger portion from the barrel portion, resulting in a negative pressure within the barrel portion of the syringe. This action is used, for instance, in dentistry to check for blood in the syringe upon aspiration to sure that anesthetic solution is not deposited into a blood vessel.

It has been realized that the standard injection device, such as a syringe, while adequate for many purposes, has certain shortcomings, especially for certain uses and users. For example, in situations where numerous injections are required an operator can become fatigued or sore from the use of a standard syringe. This can be especially problematic when relatively small volumes are being injected, when multiple injections are made in a relatively short period of time, when the syringe itself is relatively small, or when the precision of the locations of the injections is important (frequently resulting in the user gripping the syringe more tightly). While there are numerous examples of where this can occur (which are not limited to the medical profession or medical treatment), this issue can be especially prominent in the administration of substances such as dermal fillers in cosmetic treatments where many, if not all, of the above issues can be involved at once. In some of the embodiments disclosed herein, these and other considerations have resulted in various ergonomic syringes.

It is an aspect of the present invention to allow the single-handed pushing and aspiration of a syringe.

Certain syringes in the prior art attempt to address the ability to aspirate a syringe with a single hand by providing a closed loop thumb grip, into which a user disposes their thumb for actuation of the syringe. Sometimes such solutions further comprise closed loop finger grips which These solutions in certain scenarios can be cumbersome due to the manner in which a thumb must be disposed within the closed loop. As a result, a user may require two hands to establish a proper grip upon the syringe in a single hand prior to single-handed use. Furthermore, technologies surrounding a closed loop thumb grip often require the use of metallic materials such as stainless steel. Such limitations can result in prohibitive cost of equipment, for both the syringe as well as suitable cleaning equipment to sterilize the syringe and syringe components between uses.

It is an aspect of certain embodiments of the present invention to provide a broken loop thumb grip in order to allow a user to dispose their thumb within the thumb grip from a side aspect of the thumb grip to allow single-handed interaction with the syringe when initially picked up by the user, while permitting single-handed pushing and aspiration of the syringe.

It is a further aspect of certain embodiments of the present invention comprise a system for retrofitting of existing syringes with features for the increased dexterity, manipulation and ergonomics for the user of the syringe.

In some of the embodiments described herein, it has been appreciated that the user's comfort can be important. This can be especially relevant when numerous injections, small volumes to be injected, complex procedures (such as aspiration), or any combination thereof occur. Administering multiple injections with commonly available syringes in the prior art may result in hand fatigue or soreness due to sharp edges and non-ergonomic surfaces associated with standard syringes. Thus, in some embodiments, an ergonomic syringe can be employed for ease of use. In some embodiments, this is especially advantageous when the substance to be injected is a dermal filler.

The administration of dermal filler often involves repeated injections, aspiration, and manipulation of a syringe into different positions to administer dermal filler into different areas from different angles. The designs of commonly used syringes are configured for use in a limited range of orientation as is commonly accepted in use with a syringe.

In addition, in some embodiments, there is a significant need for devices and systems for one-handed aspiration in conjunction with injection. Preferably, such devices and systems for one handed aspiration and injection can be configured to reduce the risk of injury. It is therefore desirable to provide a convenient, accurate, comfortable system for one handed aspiration and injection. In certain embodiments, ergonomic features are configured to be repositionable following initial installation to allow a user to tailor the ergonomics of a syringe to their preferences, hand size, and specific needs.

In some embodiments, the present invention concerns devices and systems for aspiration and injection, especially a device for one-handed aspiration and injection.

A syringe is disclosed in accordance with some embodiments of the present invention. In some embodiments, the syringe has a barrel portion, a cushion positioned against said barrel portion, wherein the cushion is configured to provide cushioning when said barrel portion is operably grasped by two adjacent fingers of a same hand, and a plunger portion having a distal end adapted for insertion into a proximal end of said barrel portion.

In some embodiments, the syringe has a predefined volume of a dermal filler contained within the barrel portion. In some embodiments, the syringe is configured to inject a total volume of less than about 2 ml (0.068 fl. oz.) In some embodiments, the syringe is configured to inject a total volume of less than about 1 ml (0.034 fl. oz). In some embodiments, the cushion is integral to the syringe. In some embodiments, the cushion is detachable from the syringe.

In some embodiments, the syringe has a second cushion positioned adjacent to a proximal end of the plunger portion. In some embodiments, the syringe has a thumb grip positioned on a proximal end of the plunger portion. In some embodiments, an outer diameter of the barrel portion of the syringe is less than about 1 cm (0.394 in).

In some embodiments the thumb grip has a broken loop positioned to facilitate one-handed aspiration during operation. In some embodiments the syringe has a third cushion positioned on the thumb grip and configured to provide cushioning during one-handed aspiration. In some embodiments, the thumb grip is integral to the syringe. In other embodiments, the thumb grip is detachable from the syringe.

In some embodiments, a cushion is positioned against the barrel portion of the syringe and completely encircles the barrel portion. In some embodiments, a cushion positioned against the barrel portion has a substantially circular cross-sectional area encircling the barrel portion. In some embodiments, a cushion positioned against the barrel portion has a generally oblong cross-sectional area encircling the barrel portion and has an upper lip, a lower lip, or an upper lip and a lower lip. In some embodiments at least a portion of the cushion positioned against the barrel portion extends at least 5 mm (0.197 in) beyond the circumference of the barrel portion.

In some embodiments, the syringe is configured to inject multiple, pre-measured amounts of an injectable substance, wherein the pre-measured amount is about 0.1 ml (0.0034 fl. oz.). In some embodiments, the syringe has stops to indicate when a pre-measured amount of fluid has been injected, and the stops (e.g. indicators) are visible to an operator when the syringe is gripped between two adjacent fingers.

In some embodiments, the syringe has a flange located on a proximal portion of the barrel portion. In some embodiments, the syringe has a fourth cushion located adjacent to a flange on a proximal portion of the barrel portion, and the cushion is configured to provide cushioning when force is exerted against the flange.

In some embodiments, any one of the cushions of the syringe is integral to the syringe.

In some embodiments, any one of the cushions of the syringe is detachable from the syringe.

In some embodiments, the syringe is sterilizable.

A kit is disclosed in accordance with some embodiments of the present invention. The kit has at least two syringes, wherein each syringe has a barrel portion, a cushion positioned against the barrel portion, wherein the cushion is configured to provide cushioning when the barrel portion is operably grasped by two adjacent fingers of a same hand, a plunger portion having a distal end adapted for insertion into a proximal end of the barrel portion, and a specified amount of a dermal filler contained within the barrel portion. In some embodiments, the barrel portion contains about 1 ml (0.034 fl. oz.) or less of dermal filler.

A kit for injecting a dermal filler is disclosed in accordance with some embodiments of the present invention. The kit has at least one syringe. Each syringe has a barrel portion and a plunger portion having a distal end adapted for insertion into a proximal end of the barrel portion, a cushion configured to be positioned against the barrel portion and to provide cushioning when the barrel portion is operably grasped by two adjacent fingers of a same hand, and a specified amount of a dermal filler contained within said barrel portion. In some embodiments, a method of manipulating a syringe is provided. The method can include gripping a barrel portion of a syringe with two adjacent fingers of a same hand, wherein at least one cushion is positioned between at least one of the fingers and the barrel portion of the syringe and injecting a dermal filler.

In some embodiments, a syringe is provided. The syringe comprises, consists, or consists essentially of a barrel portion, a plunger portion comprising a distal end adapted for insertion into a proximal end of the barrel portion, and a thumb grip. The thumb grip can be positioned on a proximal end of said plunger portion. The thumb grip can comprise a strap. The strap can comprise a flexible, elastic, or flexible and elastic material. In some embodiments, the thumb grip is detachable from the syringe. In some embodiments, the strap comprises an elastic material. In some embodiments, the strap comprises, consists, or consists essentially of a first hole at a first end of the strap and a second hole at a second end of the strap. In some embodiments, at least a portion of the plunger portion is passed through both the first and second holes of the strap. In some embodiments, the proximal end of the plunger portion is larger in diameter than the diameter of the first, second, or first and second hole(s). In some embodiments, the strap comprises a material selected from the group consisting of rubber, nylon, cotton, foam rubber, neoprene, plastic, or any combination thereof. In some embodiments, the syringe further comprises a cushion positioned against said barrel portion. The cushion can be configured to provide cushioning when said barrel portion is operably grasped by two adjacent fingers of a same hand. In some embodiments, said thumb grip is integral to the syringe. In some embodiments, the strap is sized so as to allow a user's finger or thumb to fit between the proximal end of the plunger portion and a surface of the strap without stretching the strap. In some embodiments, the strap is sized so as to require the strap to be stretched in order to allow a user's finger or thumb to fit between the proximal end of the plunger portion and a surface of the strap. In some embodiments, the thumb grip consists essentially of a flexible, elastic, or flexible and elastic strap. In some embodiments, the strap consists essentially of a first hole at a first end of the strap and a second hole at a second end of the strap.

In some embodiments, a kit is provided. The kit comprises, consists, or consists essentially of a syringe comprising a barrel portion and a plunger portion comprising a distal end adapted for insertion into a proximal end of the barrel portion. The plunger portion further comprises a proximal end. The proximal end is larger in diameter than the distal end of the plunger portion. The kit further comprises, consists, or consists essentially of a strap comprising consisting, or consisting essentially of a first hole at a first end and a second hole at a second end of the strap. The strap comprises, consists, or consists essentially of a flexible, elastic, or flexible and elastic material. In some embodiments, the kit further comprises a cushion positioned against said barrel portion, wherein the cushion is configured to provide cushioning when said barrel portion is operably grasped by two adjacent fingers of a same hand. In some embodiments, strap is elastic.

In some embodiments, a method of making a syringe is provided. The method comprises, consists, or consists essentially of providing a barrel portion, a plunger portion, and a strap comprising, consisting, or consisting essentially of a first hole at a first end of the strap and a second hole at a second end of the strap. The strap comprises a flexible, elastic, or flexible and elastic material. One can insert a distal end of the plunger portion through said first hole of the strap. One can insert the distal end of the plunger portion through said second hole of the strap. One can insert said distal end of the plunger portion into a proximal end of the barrel portion.

In some embodiments, a method of using a syringe is provided. The method comprises, consists, or consists essentially of providing a syringe comprising a barrel portion and a plunger portion comprising a distal end adapted for insertion into a proximal end of the barrel portion, providing a strap that is looped around a proximal end of the plunger portion. The strap comprises, consists, or consists essentially of a first hole and a second hole. At least a part of the plunger portion is passed through the first hole and second hole. The strap is sized so as to allow a finger or thumb to be inserted between the proximal end of the plunger portion and the strap. The strap comprises, consists, or consists essentially of a flexible, elastic, or flexible and elastic material. One can insert a finger or thumb between the proximal end of the plunger and the strap, and one can aspirate the syringe by exerting a force against the strap via the finger or thumb. In some embodiments, the insertion of the finger or thumb between the proximal end of the plunger and the strap stretches the strap. In some embodiments, the stretched strap exerts a constricting force on the finger or thumb. In some embodiments, the strap, when not stretched, is between 2 (0.787 in) and 10 cm (3.94 in) in length. In some embodiments, the strap, when stretched, is between 2 (0.787 in) and 10 cm (3.94 in) in length.

Certain embodiments of the present invention comprise broken loop thumb grip, which is removably affixed to the proximal end of the plunger portion. The thumb grip may be attached to the proximal end of the plunger portion alternatively for the use of the syringe, and detached from the proximal end of the plunger portion prior to discarding the syringe.

Certain embodiments of the present invention comprise a finger flange which is removably affixed to the proximal portion of the barrel portion of a syringe. In certain embodiments, the finger flange is disposed over an existing flange at a proximal portion of the barrel portion of the syringe. The finger flange having an ergonomic form factor provides for more comfortable repeated use of the syringe. Such a finger flange is typically configured to interact with the palmar side of a user's fingers—typically the index and middle fingers—while the user interacts with the proximal portion end of the plunger portion.

Certain embodiments of the present invention comprise a finger flange which is removably affixed to a medial portion of the barrel portion of the syringe. In certain embodiments, such a finger flange is configured to be removable affixed and readjustable while disposed upon the barrel portion of the syringe. As such, the finger flange can be adjusted slidably along the length of the barrel portion of the syringe in a proximal or distal direction, allowing the user to adjust the finger flange according to the current need, comfort, and the size of their hands and/or fingers. A user may interact with the finger flange with the palmar side of their fingers, alternatively, a user may configure the finger flange to allow the placement of one's fingers between a flange at a proximal end of the barrel portion of the syringe and the finger flange. Thus, the user's palmar side of their fingers are in contact with the flange at the proximal end of the barrel portion, while the dorsal side of their fingers interact with the finger flange affixed to the medial portion of the barrel portion of the syringe. In combination with a broken thumb loop, such a configuration allows a user to alternatively push and aspirate the syringe without the need to reconfigure their grip of the syringe.

Certain embodiments of the present invention comprise a first finger flange and a second finger flange affixed to a medial portion of the barrel portion of a syringe. In such embodiments, the first finger flange and second finger flange can be configured in a number of configurations for increased functionality of the syringe. In one use case, the first finger flange and the second finger flange are axially aligned yet offset from each other. This configuration allows a user to dispose their fingers between the first finger flange and the second finger flange to allow the user to interface with the first finger flange with a palmar side of their fingers and interface with the second finger flange with the dorsal side of their fingers. Once again—in combination with a broken thumb loop, such a configuration allows a user to alternatively push and aspirate the syringe without the need to reconfigure their grip of the syringe.

In certain embodiments comprising a first finger flange and a second finger flange affixed to a medial portion of the barrel portion of a syringe, the first finger flange and the second finger flange are rotatively adjustable in relation to each other. In such use cases, the first finger flange and the second finger flange can be axially offset resulting in an X-shaped configuration. An X-shaped configuration allows a user to place the syringe upon a work surface such that portions of the first finger flange and the second finger flange contact the work surface. Furthermore, the proximal end of the plunger portion is also in contact with the work surface. Thus, the distal end of the syringe, specifically the needle and the tip of the needle, are raised off the work surface. Raising the needle off the work surface allows a user to put the syringe down without worry of contamination of the needle tip. Contamination of the needle tip can result in infections, including a blood infection. A blood infection, sometimes referred to as “sepsis,” is the result of introduction of bacteria, fungi, or parasites into the blood stream. As blood infections can be fatal, it is important that a needle of a syringe remain sterile.

These and other advantages will be apparent from the disclosure of the inventions contained herein. The above-described embodiments, objectives, and configurations are neither complete nor exhaustive. As will be appreciated, other embodiments of the invention are possible using, alone or in combination, one or more of the features set forth above or described in detail below. Further, this Summary is neither intended nor should it be construed as being representative of the full extent and scope of the present invention. The present invention is set forth in various levels of detail in this Summary, as well as in the attached drawings and the detailed description below, and no limitation as to the scope of the present invention is intended to either the inclusion or non-inclusion of elements, components, etc. in this Summary. Additional aspects of the present invention will become more readily apparent from the detailed description, particularly when taken together with the drawings, and the claims provided herein.

### BRIEF DESCRIPTION OF THE DRAWINGS

FIG. 1—certain embodiments of a syringe having a cushion positioned against the barrel portion, a plunger portion, a thumb cushion, and a flange.

FIG. 2—depicts certain embodiments having a barrel portion 20 having a cushion 90 and a flange 40 on the proximal end. The cushion 90 has a lower lip 100 and an upper lip 110.

FIG. 3—depicts certain embodiments having a closed looped flange 120 on the proximal end of a barrel portion 20 as well as other embodiments.

FIG. 4—depicts certain embodiments having a syringe 10 having a plunger portion 30 having a thumb grip 160 that includes a broken loop positioned on the proximal end.

FIG. 5—depicts certain embodiments having a plunger portion 30 having a looped thumb grip 180 positioned on the proximal end.

FIG. 6—depicts a side view of certain embodiments having a gripping device that is adapted to be placed on a proximal end of a syringe.

FIG. 7—depicts a side view of certain embodiments having a gripping device that is adapted to be placed on a proximal end of a syringe.

FIG. 8—depicts a side view of certain embodiments having a gripping device, such as depicted in FIG. 7 that is adapted to be placed on a proximal end of a syringe.

FIG. 9—depicts a bottom view of certain embodiments having the gripping device depicted in FIG. 7 that is adapted to be placed on a proximal end of a syringe.

FIG. 10—depicts a side view of certain embodiments having a gripping device that is adapted to be placed on a proximal end of a syringe.

FIG. 11—depicts a bottom view of certain embodiments having the gripping device illustrated in FIG. 10 that is adapted to be placed on a proximal end of a syringe.

FIG. 12—depicts a flow chart describing various possible steps that can be taken during some of the disclosed embodiments.

FIG. 13A—depicts a side view of a certain embodiments comprising a gripping device

FIG. 13B—depicts a bottom view of a certain embodiments comprising a gripping device

FIG. 14A—depicts a top view of a certain embodiments comprising a finger flange

FIG. 14B—depicts a side view of a certain embodiments comprising a finger flange

FIG. 14C—depicts a bottom view of a certain embodiments comprising a finger flange

FIG. 15A—perspective view of a system of certain embodiments having two finger flanges in an aligned configuration

FIG. 15B—depicts a perspective view of certain embodiments having two finger flanges in an X-shaped configuration

Throughout the figures, the same reference numerals and characters, unless otherwise stated, are used to denote like features, elements, components or portions of the illustrated embodiments. Moreover, while the subject matter of this application will now be described in detail with reference to the figures, it is done so in connection with the illustrative embodiments. It is intended that changes and modifications can be made to the described embodiments without departing from the true scope and spirit of the subject invention as defined in part by the appended claims.

### DETAILED DESCRIPTION OF VARIOUS EMBODIMENTS

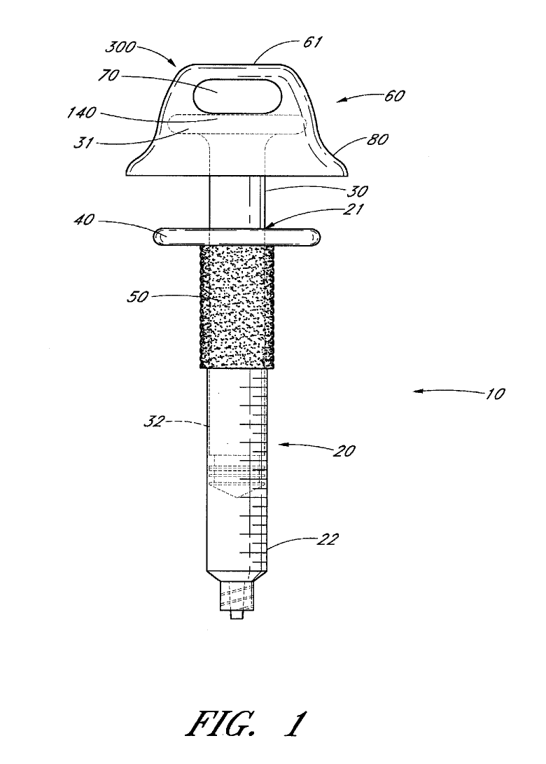

In some embodiments, the devices and methods disclosed herein allow for greater ease of use during the manipulation (e.g. injection, aspiration, or aspiration and injection) of a syringe. In some embodiments, the devices and methods allow for greater comfort during use. In some embodiments, the devices and systems disclosed herein allow the user to have an available hand during aspiration and injection to ensure, for example, patient position, tissue tautness, stability, etc. In some embodiments, the devices can be used for stable and accurate one-handed aspiration and injection. In some embodiments, the devices can be used for repeated delivery of a fluid or injectable substance. The present description first describes various embodiments of ergonomic syringes with respect to FIG. 1-FIG. 5. The description then proceeds to detail alternative embodiments and specific aspects of the ergonomic syringes, subparts, and related devices. Finally, some specific embodiments of using the various devices are disclosed.

Certain embodiments, as seen in FIG. 1, comprise a system that can be used to perform a variety of methods or procedures. The syringe 10 has a barrel portion 20 and a plunger portion 30. The plunger portion 30 has a distal end 32 adapted for insertion into a proximal end 21 of the barrel portion 20. In certain embodiments, such as illustrated in FIG. 1, the proximal end 21 of the barrel portion 20 has a flange 40 and a cushion 50 adjacent to the barrel portion 20. In some embodiments, the cushion 50 is configured to provide cushioning when the barrel portion is operably grasped by two adjacent fingers of a same hand. The cushion 50 can be made of a resilient or elastomeric material to provide additional comfort and prevent slippage during operation. In some embodiments, the cushion 50 completely encircles the barrel portion and has a substantially circular cross-sectional area encircling the barrel portion. The illustrated cushion 50 has a textured surface to prevent slipping. Preferably, the cushion 50 is formed of a resilient or elastomeric material. In some embodiments, the syringe further includes a thumb cushion 60 adjacent to the proximal end of the plunger portion 31. In some embodiments, the size and shape of the syringe 10 is particularly suitable for one-handed aspiration and injection. As will be appreciated by one of skill in the art, while the thumb cushion 60 can be depressed by the thumb, it can also be depressed or acted upon by other parts of the user's hand, such as the palm of the hand or the palmar side of a digit.

In some embodiments, such as seen in FIG. 1, the thumb cushion 60 is fitted around the proximal end 31 of the plunger portion 30, or plunger tip, and has a generally oblong cross-sectional area encircling the plunger portion. The thumb cushion 60 has a surface 61 for an operator to press against in order to depress the plunger portion. In some embodiments, the thumb cushion 60 has a lower lip 80 that can act as a flange for aspiration. The lower lip 80 can also be described as a plunger cushion flange 80. In some embodiments, rather than employing the thumb cushion 60 (having the plunger cushion flange 80 and the surface 61) by pressing on the surface 61, the proximal end 31 of the plunger portion is used as thumb grip 300. The thumb grip 300 can have a hole 70 (which need not be present when only a thumb cushion 60 is desired) generally sized to fit a thumb. The operator can insert a thumb in the hole 70 during operation, and using the thumb, either pull back the plunger portion 30 to aspirate or push down on the plunger portion 30 to inject. The thumb cushion 60 or thump grip 300 can be made of a soft or resilient material to provide additional comfort and prevent slippage during operation. In the illustrated embodiment, the thumb cushion and thumb grip have a generally oblong cross-sectional area encircling the proximal portion of the plunger portion 30.

In some embodiments, such as seen in FIG. 1, the syringe can further include a flange 40, which can serve as a grip flange for assisting with injections. As will be appreciated by one of skill in the art, while the thumb cushion and thumb grip are depicted in as extending past the proximal end 31 of the plunger portion 30, towards the distal end 32 of the plunger portion, it need not extend past the proximal end 31.

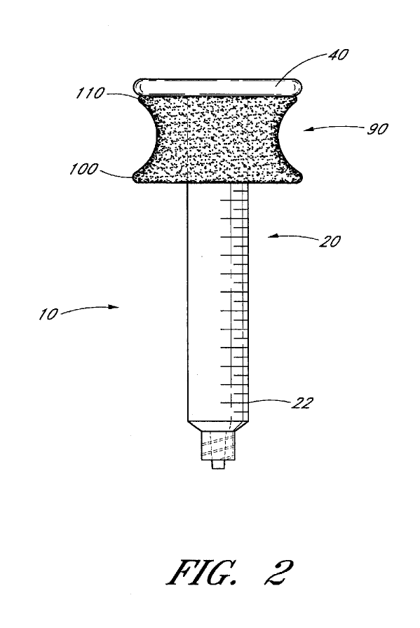

FIG. 2 shows another embodiment of a barrel portion of a syringe. In the illustrated embodiment, a cushion 90 encircles the barrel portion 20 of the syringe 10. The barrel portion 20 also has a circular flange 40. In the illustrated embodiment, the cushion 90 has a bottom lip 100 and a top lip 110. The bottom lip 100 can also be referred to as a first barrel cushion flange 100. The upper lip 110 can also be referred to as a second barrel cushion flange 110. The barrel cushion flanges 100, 110 can provide an increased surface area and features which, in some embodiments, allow for a more comfortable and secure grip when applying force during manipulation of the syringe 10. These embodiments can also allow one the option of using either one or two hands comfortably. As will be appreciated by one of skill in the art, the cushioning can provide additional comfort for the user's fingers with respect to the barrel portion 20, the flange 40, or both. Additionally, the lower lip 100, can allow for greater ease of use when the user is aspirating the syringe 10 because it can allow for a better grip on the barrel portion 20. In some embodiments, the cushion 90 is made of a soft, resilient, and/or elastomeric material. In this embodiment, the cushion 90 has a generally oblong cross-sectional area encircling the barrel portion 20. The cushion 90 can be configured to provide cushioning when the barrel portion 20 is operably grasped by two adjacent fingers of a same hand. In some embodiments, the cushioning is placed so that at least part of the cushion 90 is positioned between the user's fingers and the barrel portion 20 of the syringe 10. In some embodiments, the generally oblong cross-sectional area of the cushion 90 allows the operator to stabilize, via two adjacent fingers of a same hand, the syringe 10. The cushion 90 can be adjacent to the barrel portion 20, the flange 40, or the barrel portion 20 and the flange 40 during operation. In some embodiments, such as those seen in FIG. 1 and FIG. 2, flanges 40 in are optional and can be removed. The cushion 90 can provide the gripping surface for the user. Thus, in some embodiments, a syringe that does not have a flange, but does have a cushion, is provided. In some embodiments, the cushion is provided on the opposite side of the flange as depicted in FIG. 1 and FIG. 2. In such embodiments, the barrel can extend past the flange to allow the cushion to be attached to the syringe.

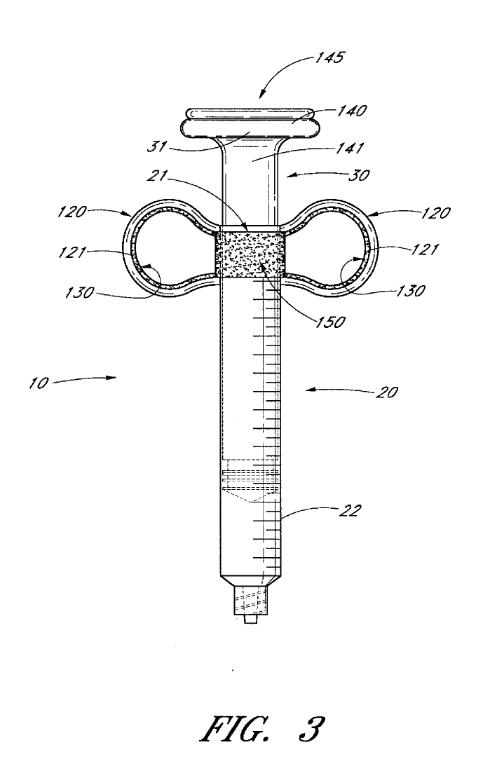

FIG. 3 shows another embodiment of a device that can be used to perform a variety of methods or procedures. In the illustrated embodiment, a closed loop flange 120 is located at the proximal end 21 of the barrel portion 20 of a syringe 10. The closed loop flange 120 has at least one, if not two, finger loops on opposite sides of the barrel portion 20. The closed loop flange 120 can be configured to be grasped by two adjacent fingers of the same hand during use of the syringe 10. As will be appreciated by one of skill in the art, while the closed loop flange 120 is depicted as closed loops, the loops need not be completely closed. In some embodiments, the closed looped flange 120 is open or broken thereby allowing for greater ease during picking up or discarding the syringe 10 by the user. As will be appreciated by one of skill in the art, while a closed loop flange 120 can provide grip security, it can take more time to grasp and/or release the device. Thus, in some embodiments, a broken or open looped flange is employed instead. This can be especially advantageous when numerous syringes are to be used and/or different syringes interchanged in relatively rapid succession (such as in a single session of injections).

Certain embodiments of the present invention comprise an open loop flange. It will be appreciated that a closed loop of any configuration comprises a 360-degree radial span. As will be appreciated by one having skill in the art, the degree to which a looped flange is “open” can vary. An open loop flange, in certain embodiments, comprises a flange having a substantially looped configuration wherein a first portion of the flange does not intersect with a second portion of the loop. An open loop flange, is typically configured such that the form of the loop terminates prior to a 360-degree radial span. Thus, a first portion of the loop does not intersect with a second portion of the loop. In some embodiments a looped flange is merely cut (and thereby allows a slight increase in flexibility to the structure); however, in some embodiments, more of the loop is removed. In some embodiments, a sufficient amount of the loop is removed, allowing a user to dispose their fingers within the loop from a lateral portion of the loop, to aid in the picking up and release of the syringe. For example, a finger's width of the loop is removed. In some embodiments, two closed loop flanges 120 (as depicted in FIG. 3) are used on the barrel portion, with the user's fingers to be placed within the two flanges. Alternatively, in certain embodiments—the closed loop flanges 120, as shown in FIG. 3, are replaced with open loop flanges (not shown). Thus, allowing a user to dispose their fingers within the open loop flange from a lateral direction. In some embodiments, more of the loop is removed, for example, to result in a structure that is similar to that depicted as the cushion flanges in FIGS. 2 (110 and 100).

In some embodiments, for example, as depicted in FIG. 3, the plunger portion 30 can have a plunger shaft 141 and a plunger tip 140 located at the proximal end 31. In some embodiments, the plunger tip 140 is formed of a resilient or elastomeric material and can have a larger diameter than the plunger shaft 14, thereby providing a flange on the plunger portion. In some embodiments, the plunger tip 140 is comprises a cushion 145 that is affixed to the plunger tip 140. The cushion 145 provides cushioning when the plunger portion 30 is pushed toward the barrel portion 20 of the syringe for injection.

In some embodiments, e.g., as depicted in FIG. 3, a cushion 121 on the inner surface 130 of each closed loop flanges 120 provides cushioning for a user's fingers during pulling and pushing actuation of the plunger portion 30. In some embodiments, a cushion 150 is adjacent to the barrel portion 20 of the syringe, thus providing lateral cushioning between a user's finger and the barrel portion 20 of the syringe. In some embodiments, the cushion 150 encircles the barrel portion. In some embodiments, the cushion 150 is in two sections, where each section is primarily located on the barrel portions that are associated with the closed loop flanges 120. In some embodiments, the cushion 150 can provide cushioning when the barrel portion is operably grasped by two adjacent fingers of a same hand.

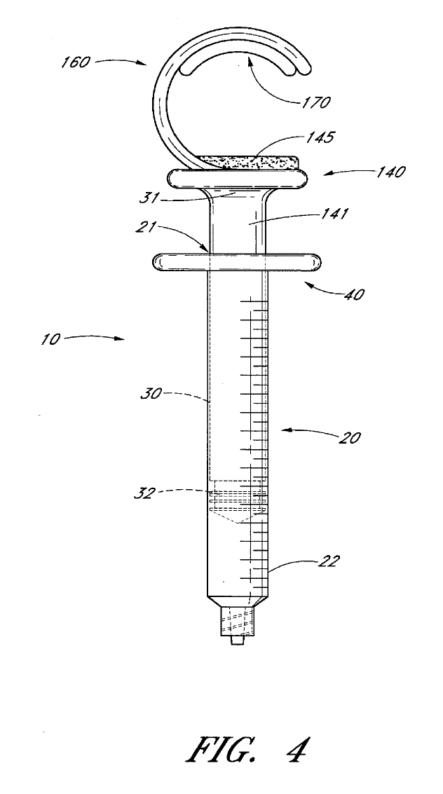

Certain embodiments, as shown in FIG. 4, are directed to a system for performing a variety of methods or procedures. A syringe 10 includes a thumb grip 160 located at the proximal end 31 of the plunger portion 30. In some embodiments, the thumb grip 160 is used to facilitate one-handed aspiration during operation. In the illustrated embodiment, the thumb grip 160 is configured in a partial or broken loop. In some embodiments, a cushion 170 is located on the thumb grip 160 to make use more comfortable. In some embodiments, the cushion 170 is made of a soft, elastomeric or resilient material and is located on the inside surface of the thumb grip 160 to provide a comfortable contact surface when the thumb is used to pull the plunger portion 30 proximally during aspiration.

The plunger portion 30 can include a plunger shaft 141 and a plunger tip 140 located at the proximal end, e.g., as depicted in FIG. 4. In some embodiments, the plunger tip 140 is formed of a resilient or elastomeric material. In some embodiments, an additional cushion 145 can be placed adjacent to the plunger tip 140. In either embodiment, depressing the plunger can be made relatively more comfortable with the cushioning provided by the cushion 145 and/or the cushion 170. Furthermore, in some embodiments, the cushion 145 and the cushion 170 are combined into a unitary cushion (not shown) spanning an interior circumference of the thumb grip 160.

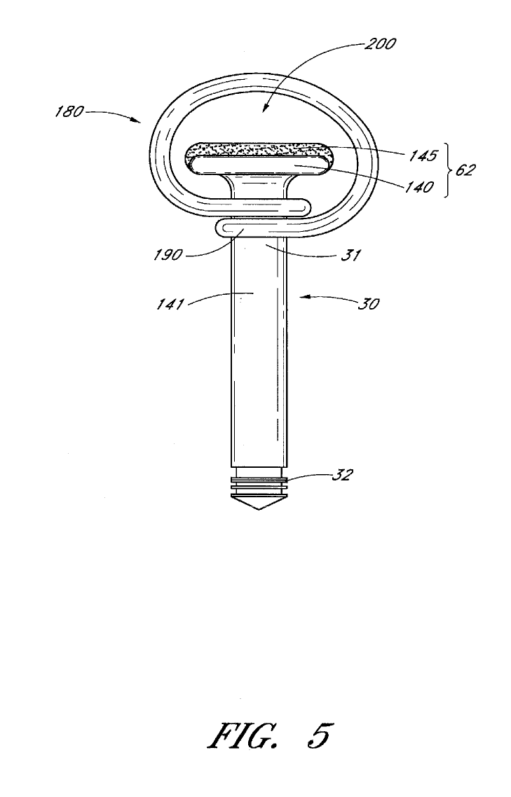

FIG. 5 depicts alternative embodiments of a plunger portion 30 that can be used to perform a variety of methods or procedures. Those of skill in the art will appreciate that the plunger portion 30 may be used with various embodiments of a barrel portion 20.

In certain embodiments, as shown in FIG. 5, a band or strap 180 is located at the proximal end 31 of the plunger portion 30 and encircles the plunger tip 140. This band, strap, or loop is configured for use as a thumb grip. For example, the band or strap can be sized or sizable to allow a user's thumb to be placed in a gap 200. As with other thumb grips, it can be used to facilitate one-handed aspiration during operation. In some embodiments, the plunger portion 30 can include a plunger shaft 141, and a plunger tip 140 located at the proximal end 31. In some embodiments, the plunger tip 140 is formed of a resilient or elastomeric material. In some embodiments, there is a cushion 145 adjacent to the plunger tip 140. The plunger tip 140 can be pushed by the thumb when injecting.

In some embodiments, as shown in FIG. 5, a band or strap 180 can be an open or closed loop. In some embodiments, the band or strap 180 can be formed of, for example, a rubber or an elastic band or strap that is wrapped around the plunger tip 140 and secured at the base 190 of the plunger tip 140 by a securing means. The securing means can be, for example, glue, a Velcro® fastener, or a hole in the band through which the plunger shaft 141 is passed. A gap 200 is present between the plunger tip 140 and the band or strap 180. A thumb or finger can be placed in the gap 200. The band or strap 180 can be used to pull the plunger portion 30 proximally to aspirate. The band or strap need not be flexible in all embodiments, although it can be made out of a flexible, elastic, or flexible and elastic material (e.g. rubber, cloth, etc.). In such an embodiment, not only can the band allow for easier aspiration, but it can also be relatively comfortable. In some embodiments, a cushion 145 is affixed to not only the plunger tip 145, but can also be affixed to the inner surface of the band 180. In some embodiments, the loop 180 can be a rigid or semi-rigid material, such as a hard plastic. In some embodiments, when the loop formed from the band or strap is smaller than the finger or thumb to be inserted into the loop, the band or strap can be elastic, providing for the extra size of the band or strap when the band or strap is stretched. In some embodiments, the stretched band or strap helps to keep the proximal end of the plunge adjacent to or in contact with the user's finger or thumb. In some embodiments, this provides for a greater degree of control over the syringe. Further variations on these embodiments are discussed in greater detail below.

In an alternative embodiment of FIG. 5, the band 180 is configured for use as or is used as a thumb cushion 62. In some embodiments, the band 180 can serve as a cushion, can aid in securing a cushion 145 (if such a cushion is present in the embodiment) to the plunger portion 30, or do both. In such an embodiment, while a gap 200 can be present, one of skill in the art will appreciate that it will likely be reduced or eliminated when the plunger is depressed into the barrel portion.

As will be appreciated by one of skill in the art, the band 180 and the cushion 145 need not be placed adjacent to the underside of the plunger tip 140. In some embodiments, one or both are located primarily adjacent to the top (proximal) section of the plunger tip 140.

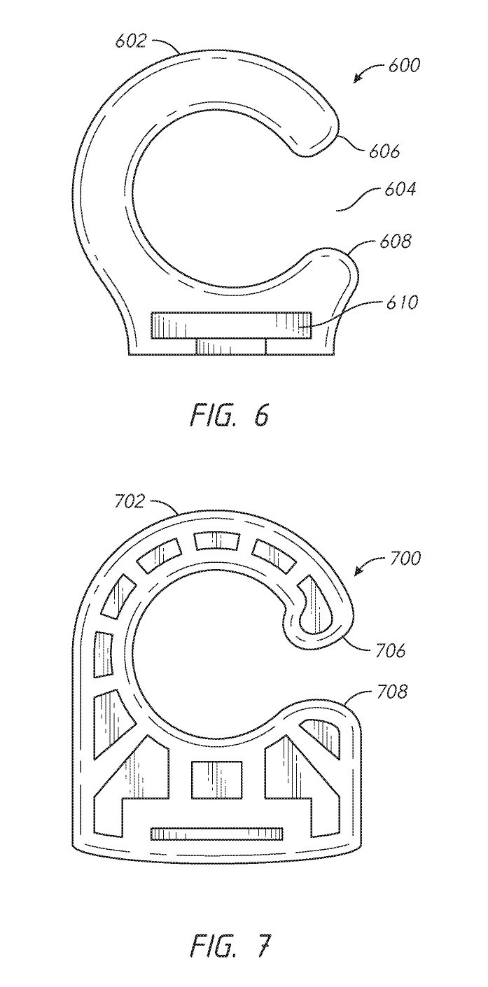

FIG. 6 depicts a side view of a gripping device 600 that is adapted to be attached (e.g., integrally or detachably) to a plunger tip (e.g., plunger tip 140 illustrated in FIG. 1). The gripping device 600 includes an open loop portion 602 that is sized and configured to operatively receive a user's thumb so that the user may move the plunger (e.g., to aspirate and inject) when the gripping device 600 is attached to a plunger tip. The open loop portion 602 may be fabricated from resilient materials for example. The opening 604 between a first end 606 and a second end 608 of the open loop portion 602 may advantageously enable a user to place their thumb within the open loop portion 602 from the side of the device 600, as opposed to being forced to insert their thumb through a closed loop. The device 600 also includes an attachment slot structure 610 for slidably engaging the flange of a plunger tip. An exemplary attachment slot structure is discussed in more detail below with respect to FIG. 9.

Certain embodiments, such as shown in FIG. 7, comprise a gripping device 700 that is adapted to be attached (e.g., integrally or detachably) to a plunger tip (e.g., plunger tip 140 illustrated in FIG. 1). As compared to the gripping device illustrated in FIG. 6, the gripping device 700 includes an enlarged portion 707 at the first end 706. The enlarged portion 707 further facilitates engagement of the thumb (or other finger) of a user with the gripping device 700 thus preventing a user's thumb (or finger) from inadvertently slipping out of the of open loop.

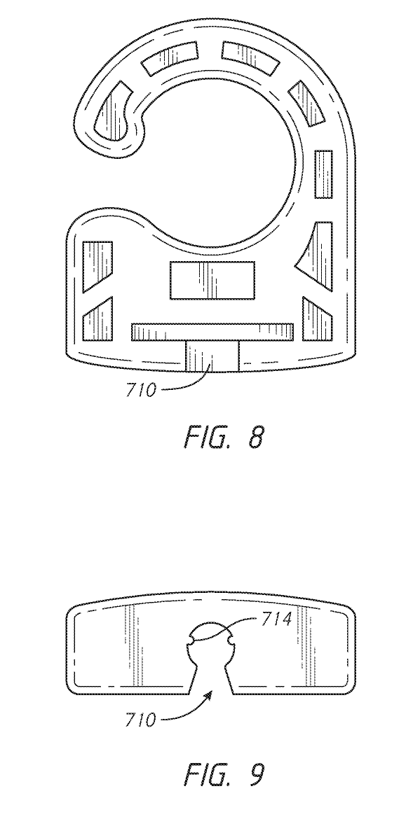

An alternate side view (i.e., opposite the side view of FIG. 7), shown in FIG. 8, demonstrates the gripping device 700 from another side. As shown, in certain embodiments the gripping device 700 further comprises an attachment slot structure 710 that is adapted to slidably engage the flange of a plunger tip, e.g., so that the gripping device is readily attachable and detachable from the plunger tip.

In certain embodiments, as shown in FIG. 9 (depicting a bottom view of the gripping device 700), a gripping device 700 includes an attachment slot structure 710 that has a “keyhole” shaped recess to accommodate and grip plunger shafts of varying diameter. That is, the attachment slot structure may include an opening that narrows toward the central portion of the bottom surface of the gripping device, and then opens to a generally rounded central portion through which a plunger shaft may be accommodated. The slot structure may also include rotation restriction features, such as two knobs 714 that restrict rotational movement of the gripping device 700 with respect to the plunger.

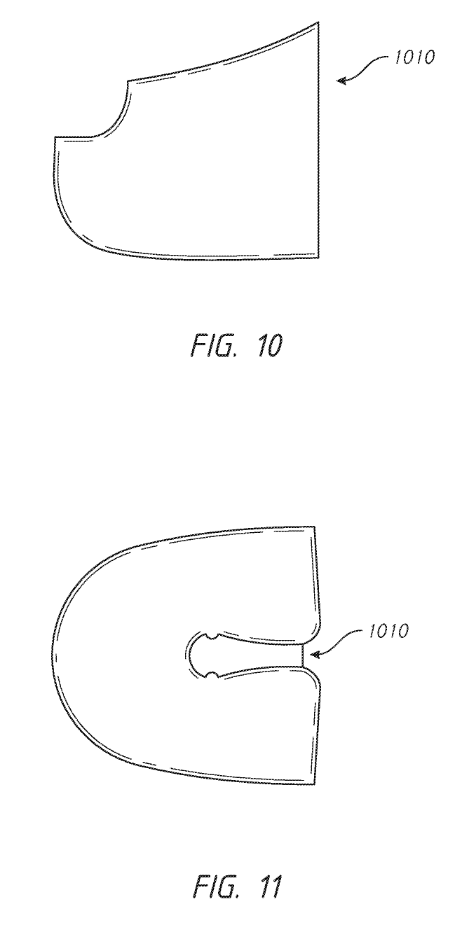

In certain embodiments, as seen in FIG. 10-FIG. 11, a gripping device 1000 is generally configured to receive the tip of a user's thumb or finger. The gripping device 1000 is generally in the shape of a user's finger, such as a user's thumb. In this way, a user may insert his/her thumb into an open end of the gripping device 1000. The gripping device 1000 may be attached to a plunger tip using the attachment slot structure 1010, which may also be in the general shape of a keyhole, as is described above with respect to FIG. 9.

As will be appreciated by one of skill in the art, any of the above items or figures can be appropriately combined with other items or figures for alternative embodiments. For example, a plunger portion in one figure can be exchanged for any of the other plunger portions in the other figures. Similarly, for example, the barrel portion of FIG. 1 can be replaced with the barrel portion depicted in FIG. 2 or the plunger portion in FIG. 3 can be used in place of the plunger portions in the other figures. The cushion 50 can be used in the embodiments depicted in FIG. 3 or FIG. 4, etc. Similarly, while many of the embodiments described in greater detail below are described in regard to specific applications, one of skill in the art will appreciate that the various embodiments can readily be applied in other situations as well. Thus, the various combinations, unless specifically noted as such, are not to be taken as limiting. The following section generally describes various additional or alternative embodiments and aspects of various embodiments of ergonomic syringes and their use.

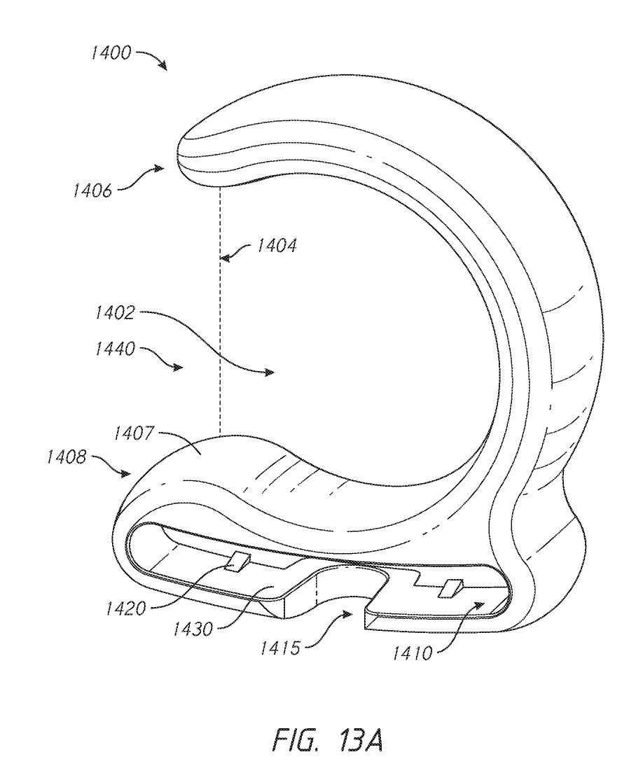

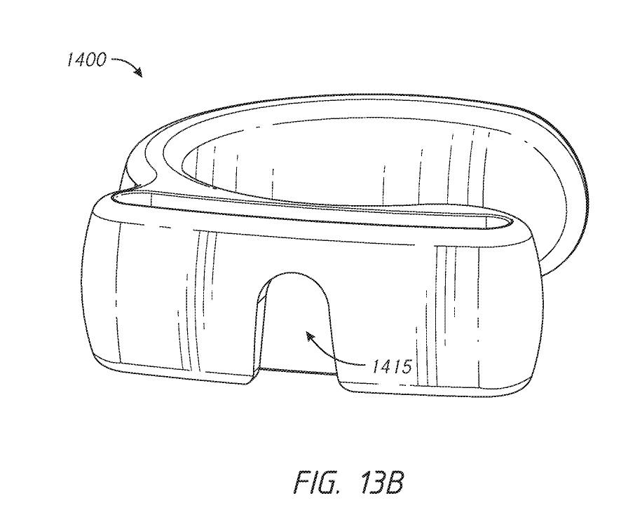

In certain embodiments, as shown in FIG. 13A-FIG. 13B, a gripping device 1400 comprises an open loop structure with a first end 1406 and a second end 1408 and an open loop portion 1402. The gripping device of certain embodiments comprises an enlarged portion 1407 configured to help retain a user's thumb within the open loop portion 1402 of the gripping device. In certain embodiments, the gripping device 1400 comprises a slot structure 1410 enabling a user to dispose the proximal portion of a plunger within the slot structure 1410 for purposes of affixing the gripping device 1400 to the plunger. In certain embodiments, the slot structure 1410 further comprises a recess 1415 configured to receive a plunger shaft 141 (FIG. 5) of a plunger. Thus, the recess 1415 permits further engagement of the slot structure 1410 with a proximal portion of a plunger portion, such as a plunger tip 140 (FIG. 4). In certain embodiments, a retention feature 1420 protrudes from a surface 1430 of the slot structure. The retention feature 1420 is configured to retain and/or lock a plunger tip 140 within the slot structure 1410. In certain embodiments, a retention feature 1420 comprises a wedge aspect such that the plunger tip 140 is easily traverses into the slot structure 1410, but the plunger tip 140 meets resistance when attempts are made to remove the plunger tip 140 from the slot structure 1410.

It will be appreciated that an open loop structure, as seen in FIG. 13A-FIG. 13B, is configured in certain embodiments to allow a user to pass their finger through the opening 1404 from a lateral approach 1440 to the loop structure versus orthogonally to the open loop structure. In certain embodiments, an open loop structure comprises a first end 1406 extending away from a second end 1408 forming an open loop form of less than 360-degrees while remaining within the spirit and the scope of the present invention. However, it may be desired in certain embodiments for the first end 1406 can extend between more than 180-degrees, but less than 315-degrees from the second end 1408.

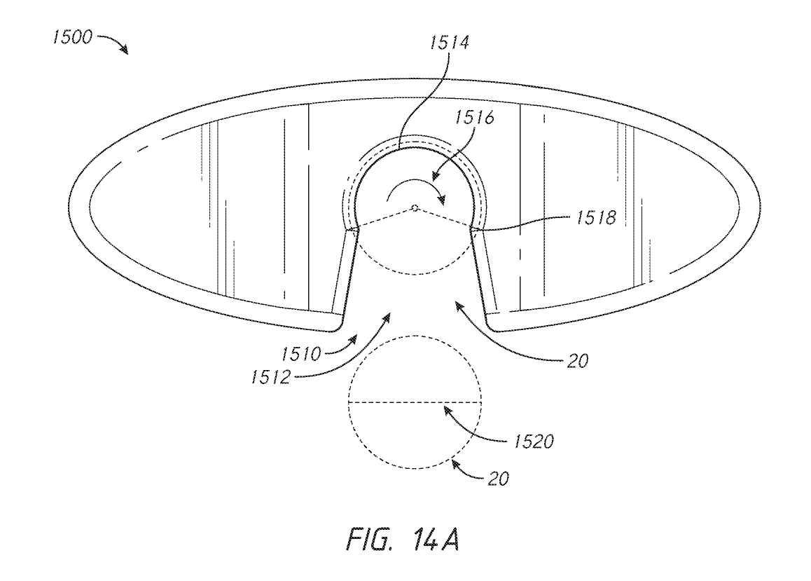

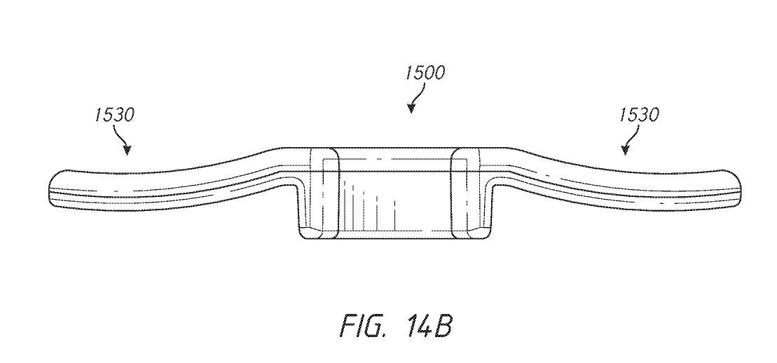

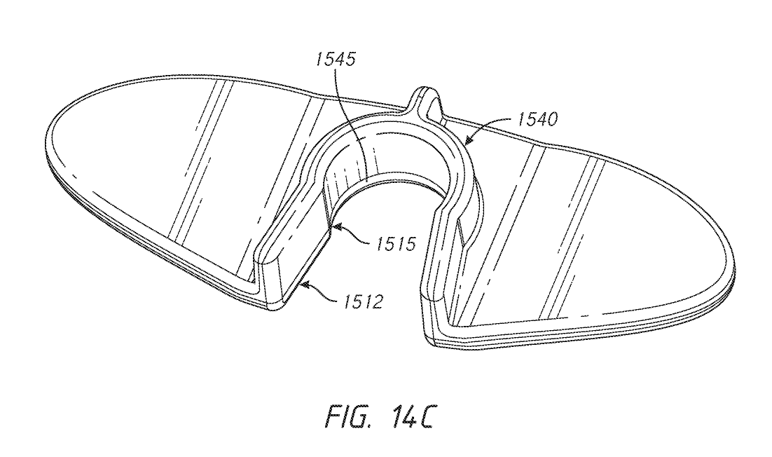

Certain embodiments of the present invention comprise a finger flange 1500, shown in FIG. 14A-FIG. 14C, which is configured to affix to the barrel portion 20 (FIG. 1-FIG. 4) of a syringe. Such a finger flange 1500 of certain embodiments is configured to be removably affixable to the barrel portion 20 of a syringe, while certain embodiments the finger flange 1500 is slidably adjustable along the barrel portion 20 of the syringe.

A finger flange of certain embodiments, shown in FIG. 14A for example, comprises an attachment feature 1510 configured for the attachment to the barrel portion of a syringe. The attachment feature of certain embodiments comprises an engagement feature 1512 configured to receive the barrel portion of the syringe. As shown, the engagement feature 1512 features a tapered profile narrowing toward a collar portion 1514 of the attachment feature. The attachment feature of certain embodiments comprises a collar portion 1514 which is configured to have an engineering fit with the barrel portion 20 of the syringe. It will be appreciated by those skilled in the art, that an engineering fit as referred to herein surrounds the fitment of a first element with a second element with geometric dimensioning and tolerance, such as a clearance fit, transition fit, or interference fit. In certain embodiments, the collar portion is configured to mate with the barrel portion of syringe wherein the collar portion has a substantially similar profile to the barrel portion of the syringe. In certain embodiments, the collar portion 1514 comprises a radial profile. In certain embodiments, the radial profile traverses a path comprising an arc angle 1516 of up to 180-degrees. However, it is desired in certain embodiments for the arc angle 1516 traverse in excess of 180-degrees, thus creating a snap-point 1518. As shown in FIG. 14A for instance, an attachment feature 1510 is passively constrained to the barrel portion 20 of a syringe once a midline 1520 of the barrel portion traverses past the snap-point 1518. Certain embodiments comprise a first snap point 1518 and a second snap point 1519 having a spacing 1519 less than the width 1520 of the barrel of the syringe.

In certain embodiments, as shown in FIG. 14B, a finger flange 150 comprises a first wing 1530 and a second wing 1530 disposed laterally from an attachment feature. The wings 1530 are configured ergonomically to interface with the palmar side or dorsal side of a user's fingers.

In certain embodiments—as shown in FIG. 14C showing a bottom view of a finger flange 1500, —finger flange 1500 comprises a stiffening structure 1540 for the added rigidity and durability of the finger flange. The stiffening structure 1540 of certain embodiments comprises a rib structure 1545, extending away from the collar portion 1514—consistent with a first surface 1550 of the finger flange, thereby providing increased thickness for particular portions of the finger flange 1500. In certain embodiments, a rib structure extends away from the collar portion 1514 in efforts to provide increased rigidity and durability to portions of the finger flange 1500 which undergo increased stress factors. It will be appreciated that portions of the rib structure 1545 can be strengthened by increasing the thickness or height of the rib structure. Alternatively, the rib structure can be weakened—for instance for a more flexible finger structure or for material efficiency in manufacturing—by decreasing the thickness or height of the rib structure.

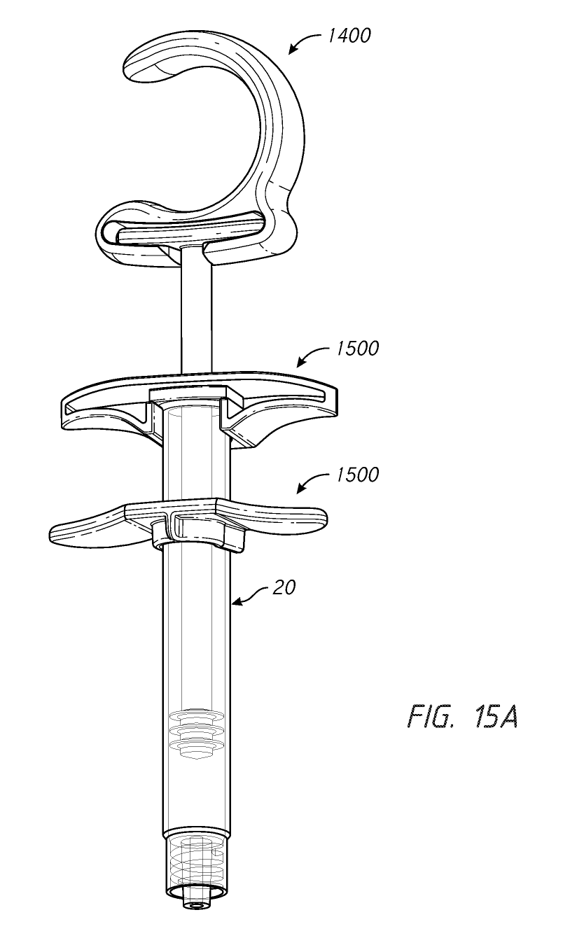

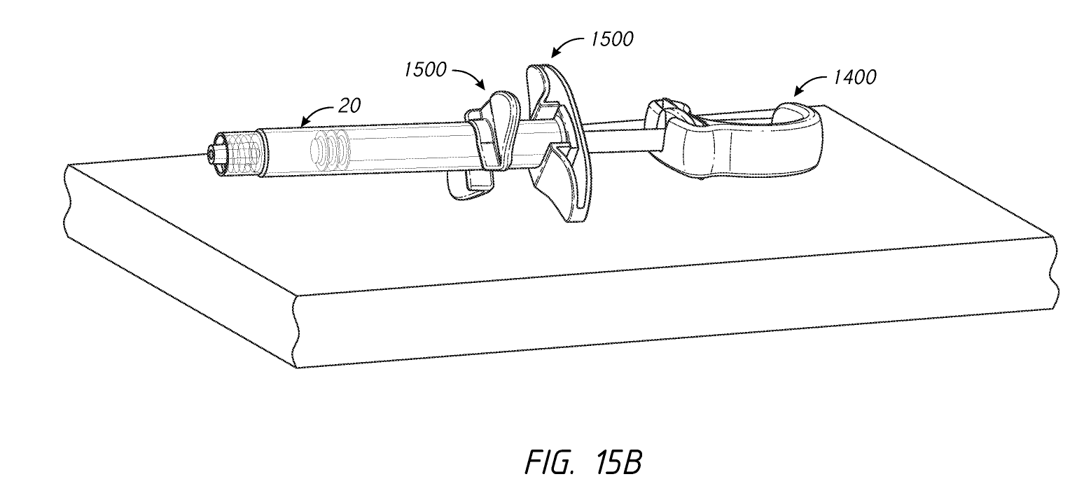

In certain embodiments comprising a system, seen in FIG. 15A-FIG. 15B, a first finger flange 1500 and a second finger flange 1500 are affixed to the barrel portion 20 of a syringe. In certain embodiments, a first finger flange 1500 is affixed to a flange 40 is at the proximal end of the barrel portion 20 of a syringe. Certain embodiments of a system comprise a gripping device 1400 affixed to the plunger tip 140 at the proximal end of the syringe. It will be appreciated that any number of finger flanges 1500 may be used in such systems as desired. A user can configure the finger flanges 1500 in any configuration desired. Two such examples are demonstrated in FIG. 15A and FIG. 15B. As shown in FIG. 15A, a user may align the finger flanges for ease of use for alternatively aspirating and injecting. Alternatively, as shown in FIG. 15B, a user may misalign the finger flanges creating an X-shaped configuration to allow the user to easily put the syringe down and pick the syringe up with a single hand and not risk contamination of the needle.

In certain embodiments, a first finger flange is integrally attached to a second finger flange, wherein the first finger flange is axially offset from the second finger flange to form a single unitary finger flange having a x-shaped profile.

### ADDITIONAL VARIATIONS OF THE EMBODIMENTS

Any or all of the following embodiments can be used in isolation or in any or all of the above described embodiments.

In some embodiments, a syringe configured for comfortable, one-handed aspiration and injection is disclosed. In some embodiments, the syringe is configured for one-handed aspiration prior to injection to ascertain that the needle is not in a vessel or other inappropriate anatomical region for the procedure. In some embodiments, the syringe is configured for immediate injection post-aspiration without moving or losing the syringe or needle placement. In some embodiments, the devices and systems disclosed herein provide means for improved accuracy in aspiration and injection compared to two handed aspiration, which can cause significant displacement of the needle.

In some embodiments, the syringes (or parts thereof) disclosed herein are single-use syringes. The syringe can have a barrel portion and a plunger portion having a distal end adapted for insertion into a proximal end of the barrel portion. The barrel portion can include a generally cylindrical, longitudinal passage extending through the barrel portion. In some embodiments, the outer diameter of the barrel portion is less than about 1 cm (0.393 in), for example, 1-0.9, 0.9-0.8, 0.8-0.7, 0.7-0.6, 0.6-0.5, 0.5-0.4, 0.4-0.3, 0.3-0.2 cm or smaller. In some embodiments, the inner diameter of the barrel portion is less than about 1 cm (0.394 in), for example 1-0.9, 0.9-0.8, 0.8-0.7, 0.7-0.6, 0.6-0.5, 0.5-0.4, 0.4-0.3, 0.3-0.2 cm or smaller. In some embodiments, the syringe is small enough to be readily manipulated in one hand and allow for visibility of the barrel portion of the syringe during the manipulation. In some embodiments the inner and outer diameter of the barrel portion can be significantly wider, as it can be desirable to shorten the distal to proximal length of the syringe to accommodate new techniques developed as a result of the greater control and ease of aspiration in using these devices. In some embodiments the inner and outer barrel diameters may be as wide as 2.5 cm (0.984 in), for example, 2.5-2, 2-1.5, 1.5-1, or smaller. In some embodiments, the volume in the syringe is still approximately 2 ml (0.068 fl. oz.) or less even when the syringe is wide.

In some embodiments, the distal end of the barrel portion has an axially extending tip section which can be configured for connection to a Luer lock fitting. The barrel portion can be constructed of a variety of suitable materials, including, for example, glass, metal, and plastic. In some embodiments, the material has similar characteristics of expansion and compression, in regard to temperature changes as the syringe. This can help maintain the integrity of the syringe and Luer-lock fitting during storage or sterilization. Additionally, in some embodiments, the distal end of the barrel has an axially extending tip section for connection directly to a needle, without a Luer-lock fitting.

The plunger portion 30 can include a plunger shaft 141, and a plunger tip 140 located at the proximal end 31. In some embodiments, the plunger tip 140 is formed of a resilient or elastomeric material. In some embodiments, the distal end of the plunger portion includes a resilient or elastomeric material.

In some embodiments the syringe contains a predefined volume of a substance to be injected, e.g. a flowable substance such as a suspension or liquid. In some embodiments, the substance is one that can be used in cosmetic applications. In some embodiments, this can be a dermal filler, such as RESTYLANE® dermal filler. In some embodiments, the syringe is configured to inject a total volume of less than about 2.0 ml (0.068 fl. oz.). In some embodiments, the syringe is configured to inject a total volume of less than about 1 ml (0.034 Fl. oz.). In some embodiments, the syringe contains 2-1.5, 1.5-1, 1-0.5 or less ml of the substance to be injected within the syringe. In some embodiments the substance is botulinum toxin, e.g. botulinum toxin A.

In some embodiments, the syringe is configured to allow for multiple injections of a pre-measured amount of fluid. The pre-measured amount of fluid that can be injected can be in the range of about 0.05 (0.0017 fl. oz.) to 0.5 ml (0.017 fl. oz.). In some embodiments, the pre-measured amount of fluid to be administered is about 0.1 (0.0034 fl. oz.) ml to 0.25 ml (0.0085 fl. oz.). In some embodiments, the syringe has stops to indicate when an amount of fluid has been injected. In some embodiments, the stops are visible to an operator when the syringe is gripped between two adjacent fingers.

In some embodiments, the syringe has at least an adaptor such as a cushion, flange, flange grip, thumb grip, loop, or any combination thereof. The adaptors can provide for a syringe that can be manipulated with greater ease. In some embodiments, the adaptors allow for a more secure grip when applying force during aspiration or injection. In some embodiments, the adaptors can also make the syringe more comfortable to the operator during use. The adaptors can be for single use or multiple use. In some embodiments, the adaptor is integral to the syringe. In some embodiments, the adaptor is detachable from the syringe. In some embodiments, while the adaptor is not fixed to the syringe, it is located adjacent to the syringe. The adaptor can be attached to the syringe by a variety of means, including, for example without limitation Velcro® fastener, snaps, stretch fit, clamps, glue, tape, rubber cement, adhesives, magnet, mating grooves, interlocking parts, friction, suction, pins, ties, and/or compression fit. In some embodiments, the adaptor is machined to closely fit the syringe. In some embodiments, the adaptor can be sterilized.

Cushions can be positioned in various locations on, against, or associated with the syringe (or other adaptors) to provide a surface for a more secure grip when applying force during aspiration or injection and/or make using the syringe more comfortable. For example, the cushions can be positioned against the barrel portion 20 of the syringe, adjacent to a proximal end of the plunger portion of the syringe, on a thumb grip attached to the plunger portion, adjacent to a flange located on a proximal portion 21 of the barrel portion of the syringe, or any combination of locations thereof. In some embodiments, the syringe has a cushion positioned against the barrel portion 20, wherein the cushion encircles the barrel portion. In some embodiments, the cushion is clipped to the barrel portion 20. In some embodiments, the cushion extends part way down the barrel from proximal portion to a more distal portion without encircling the barrel.

As will be appreciated by one of skill in the art, in light of the present disclosure, the shape and extent to which a cushion covers a barrel portion or other structure can vary depending upon a number of factors. In some embodiments, the cushion is placed only where needed to distribute forces applied by the fingers. In some embodiments, the cushion completely encircles a barrel portion. In some embodiments, the cushion covers the entire outer surface of the barrel portion (or other structure). In some embodiments, less of the structure is covered. For example, in various embodiments, less than 100%, 100-90, 90-80, 80-70, 70-60, 60-50, 50-40, 40-30, 30-20, 20-10, 10-5, 5-1% or less of the outer surface area of the barrel portion (or other structure) is covered with a cushion. The cushion can be a single cushion or can be separated into numerous parts. For example, the cushion could involve two cushions, each on opposite sides of the barrel portion. Any number of cushions could be used, for example, 1, 2, 3, 4, 5, 6, 7, 8, 9, 10, or more cushions can be used.

In some embodiments, the cushioning is removable from the syringe and can be used on different syringes. Thus, in some embodiments, the cushioning can be a removable sleeve that can be used on different syringes. As will be appreciated by one of skill in the art, various embodiments of the cushions described herein can be elastic. In some embodiments, the elastic cushions are attached to the syringe via their elastic properties (for example, the cushion 50, in FIG. 1 can stay on the syringe because it exerts a constricting force on the barrel of the syringe). Even in such embodiments, in some embodiments, the cushion can still be removable from the syringe. When a cushion is removable from a syringe and covers some of the barrel portion, it can be referred to as a “sleeve” or “sleeved” cushion.

In some embodiments, the cushions can be worn by the user to make using the syringe more comfortable. For example, cushions can be placed on at least a portion of the thumb, fingers, or any combination of thumb and fingers, that come into contact with the syringe or syringe grips. In some embodiments the cushions may be a part of the glove worn by the injector. For example, in some embodiments, the glove is padded in the appropriate locations. For example, the glove can have padding located in positions so that when the syringe is gripped, the padding is placed between the user's fingers or hand and the syringe. In some embodiments, the padding is limited to such locations, so that padding is not present elsewhere in the glove (or only minimally present).

The cushions can be for single use or multiple use. In some embodiments, the cushion is integral to the syringe. In some embodiments, the cushion is detachable from the syringe. The cushion can be attached to the syringe by a variety of means, including, for example without limitation Velcro® fastener, snaps, stretch fit, clamps, glue, tape, rubber cement, hooks, adhesives, magnet, mating grooves, interlocking parts, friction, suction, pins, ties, compression fit. In some embodiments, cushion is machined to closely fit the syringe. The cushion can be sterilized.

The cushion can be made of a variety of materials suitable for providing a relatively soft, relatively comfortable contact surface (for example, in contrast to the glass or hard plastic of the syringe). For example, the cushion can be made of rubber, silicone, cotton, plastic, polymer, various gels, latex, GORE-TEX® (PTFE, EPTFE), thermoplastic elastomers (TPE), neoprene, nitrites, etc. Materials also include the materials used in Dr. Scholl's® brand of gel inserts for shoes or materials of similar rigidity or compressibility. Examples of such materials are disclosed in U.S. Pat. Nos. 7,140,126 and 6,598,321 (the gel disclosures of which are incorporated herein by reference) and include non-foam elastomers such as the class of materials known as viscoelastic polymers or silicone gels, the entirety of which are incorporated herein by reference for all purposes. In some embodiments, such gels show high levels of damping when tested by dynamic mechanical analysis performed in the range of −50° C. to 100° C. In some embodiments, the mechanical properties of the gel can be more viscous than elastic and the gel provides a high energy absorption. Additional gels that can be used are thermoplastic elastomers (elastomeric materials), such as materials made from polymeric families, including but not limited to the Kraton family of styrene-olefin-rubber block copolymer, thermoplastic polyurethane, thermoplastic polyolefin, polyamide, polyurea, polyester and other polymer material that reversibly soften as a function of temperature. Kraton block copolymer of styrene/ethylene-co-butylene/styrene or styrene/butadiene/styrene with mineral oil incorporated into the matrix as a plasticizer can also be used in a cushion.

Preferably, the cushions are made of a material providing a resilient or yielding surface. In some embodiments, the cushions provide comfort and/or prevent slippage when using the syringe. In some embodiments, the cushions are made of rubber and have a textured surface. The textured surface can provide a surface for a more secure grip when applying force during aspiration or injection. The textured surface can also provide for greater comfort during manipulation of the syringe. In some embodiments the cushion is or comprises an elastic component.

The cushions can be of any thickness suitable for providing a comfortable surface on the syringe or adaptor. In some embodiments, the cushion is between about 1 mm (0.039 in) to about 15 mm (0.591 in) thick. In some embodiments, the cushion has a thickness of about 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15 mm or any amount on either side of or defined between the aforementioned thickness values. A cushion can have varying thicknesses. For example, a portion of the cushion can be about 0.5 cm (0.197 in) thick, and another portion of the cushion can be about 1 cm (0.394 in) thick.

As will be appreciated by one of skill in the art, in light of the present disclosure, the cushions can be yielding, compressible, cushioning, and/or deformable to a sufficient extent to provide for some increase in comfort, grip, or both (as desired or required). As will be appreciated by one of skill in the art, the amount of the above characteristic, such as compressibility, will vary depending upon the particular location of the cushion and the various factors involved in the use that the syringe is to be implemented in. For example, applications in which precision of volume injected is less important (or mitigated by other factors) will allow for a greater amount of deformability to be present. Similarly, applications where precision of the location of the injections are of relatively less importance will also allow materials with greater deformability to be used. When such factors are of more importance, one would generally favor cushions with less deformability. Thus, depending upon user preference and the actual use of the device, the cushions can have a compressibility of any amount, for example from less than 1% to 99% or more, including 1-2, 2-3, 3-5, 5-8, 8-10, 10-15, 15-20, 20-30, 30-40, 40-50, 50-60, 60-70, 70-80, 80-90, 90-95, 95-99 or more during the use of the syringe. This amount can be determined by measuring the initial height or thickness of a cushion while it is not being used (e.g., while no force is being applied) and comparing it to the height or thickness of the cushion while it is being used. Thus, a cushion that changes thickness by 50% when it is being used has a compressibility of 50%. Rubber and similar substances can be measured for “softness” according to Shore (Durometer) tests. In some embodiments, the cushion is between 20 and 95 on Shore A scale or up to about 50 on a Shore D scale. In some embodiments the cushion is up to 60 on the Shore A scale. In some embodiments, the cushion is below 40, 30, or 20 on the Shore A scale. However, other values above and below these can be used in some embodiments. As will be appreciated by one of skill in the art, substantial comfort can be obtained with minimal compressibility. For example, the cushion can compress less than 5%, 5-2, 2-1, 1-0.5, 0.5-0.1, or less. Additionally, not all of the cushions have to be compressible or deformable. In some embodiments, the padding or cushions simply add comfort for the user. In some embodiments, the cushion adds comfort for the user without requiring that the cushion be deformable or measurably compressible.

In some embodiments, the cushions are at least partially transparent. In some embodiments, the cushions are sufficiently transparent to allow a user to view the amount of substance being administered when the plunger is depressed. However, depending upon the placement of the cushion, the amount of transparency can vary and can be less than 100%, for example, 100-90, 90-80, 80-70, 70-60, 60-50, 50-40, 40-30, 30-20, 20-10, 10-1, or less. In some embodiments, the cushions are sufficiently transparent so as to allow a user to read the volume indicators on the syringe and/or identify the tip of the plunger with respect to these volume indicators.

In some embodiments the cushion has a substantially circular cross-sectional area encircling the barrel portion or plunger portion of the syringe. In other embodiments, the cushion has a generally oblong cross-sectional area encircling the barrel portion or plunger portion of the syringe. The cushion can have an upper lip, a lower lip, or an upper lip and a lower lip. The lips can provide increased surface area and features when desired to enable a more secure grip when applying force during injection and or aspiration. In some embodiments, at least a portion of a cushion positioned against a barrel portion extends at least about 0.5 cm (0.197 in) beyond the circumference of the barrel portion. In some embodiments, at least a portion of a cushion adjacent to a proximal end of the plunger portion of the syringe extends at least about 0.5 cm (0.197 in) beyond the circumference of the plunger portion. In some embodiments, the cushion is deformable, has shape memory, or compressible.

In some embodiments the syringe includes a thumb grip 300, 160, 180, e.g. a loop or ring sized (or sizable) for a thumb. The thumb grip can be used to facilitate one-handed aspiration during operation. In some embodiments, the thumb grip is located at a proximal end 31 of the plunger portion 30 of a syringe 10. The thumb grip can be in a closed loop or in an open configuration. In some embodiments, the thumb grip is a broken loop. In some embodiments, the thumb grip includes a cushion having a hole generally sized to fit a thumb. In some embodiments, the thumb grip includes an elastic band configured to fit around the plunger tip. In some embodiments, the elastic band encircles the plunger tip. In some embodiments the band is hard plastic and attaches through interlocking compression design.

In some embodiments, the thumb grip can be rigid or flexible. The thumb grip can be made of a variety of materials, including without limitation, rubber, cloth, plastic, elastic, glass, metal, neoprene, GORE-TEX®, silicone, TPE, nylon, cotton, foam rubber, the materials discussed above in regard to the cushion, or any combination thereof. In some embodiments, the thumb grip functions as a cushion. In some embodiments, the thumb grip functions as an aspirating loop, so as to allow one to pull the plunger out of the barrel. As will be appreciated by one of skill in the art, while this is called a “thumb” grip, other fingers can be used in this grip.

In some embodiments, a cushion is located on the thumb grip to make use more comfortable. In some embodiments, the cushion is configured to provide a comfortable surface while pushing down on the plunger portion. In some embodiments, the cushion is configured to provide a comfortable surface when the thumb grip is used to pull back on the plunger portion. In some embodiments, the thumb grip is used to pull the plunger portion proximally to aspirate. In other embodiments, the thumb grip is used to push the plunger portion distally to inject. In other embodiments, the thumb grip is used to both push the plunger portion distally to inject and alternately to pull the plunger proximally to aspirate.

The thumb grip can be for single use or multiple use. In some embodiments, the thumb grip is integral to the syringe. In some embodiments, the thumb grip is detachable from the syringe. The thumb grip can be attached to the syringe by a variety of means, including, for example without limitation Velcro® fastener, snaps, stretch fit, clamps, holes, glue, tape, rubber cement, hooks, adhesives, magnet, mating grooves, interlocking parts, friction, suction, pins, ties, compression fit. In some embodiments, the thumb grip is machined to closely fit the syringe. In some embodiments, the thumb grip can be sterilized or sterilizable.

In some embodiments, the thumb grip includes, comprises, consists, or consists essentially of, a flexible, elastic, or flexible and elastic strap that is or can be configured to fit around the distal end of the plunger 32 and then slid up to the proximal end 31 of the plunger portion to its position of use. The strap can be made of a variety of materials, including without limitation, rubber, cloth, plastic, elastic, glass, metal, neoprene, GORE-TEX®, silicone, TPE, nylon, cotton, foam rubber, or any combination thereof. In some embodiments, the strap encircles the plunger tip.

In some embodiments, the strap is sized so that, when attached to the proximal end of the plunger, it forms a loop that encompasses the plunger tip 140 and that is the size of a user's finger or thumb. In some embodiments, the strap forms a loop that is smaller than the size of the user's finger or thumb that will manipulate the plunger. However, as the strap can be elastic, the strap can stretch to encompass the user's finger (which can include thumb) and stably associate that plunger with the user's finger.

In some embodiments, the strap can have at least one hole through which the distal end of the plunger portion of a syringe can be passed, thereby attaching the strap on the plunger portion of the syringe. For example, the strap can be attached to the plunger portion by insertion of a distal end of the syringe through a hole at each end of the strap, thus forming a loop around a proximal end of the plunger portion (for example, FIG. 5). The loop is preferably generally sized to fit a thumb or finger (if the material is elastic, then the stretched loop can be the part that is appropriately sized). The operator can insert a thumb or finger in the loop during operation, and either pull back the plunger portion to aspirate, via the strap, or push down on the plunger portion to inject.

The strap can be of any length suitable for allowing a user's thumb or finger to be inserted between the proximal end of the plunger portion and the strap (in either the stretched or unstretched arrangements). In some embodiments, the strap is between about 2 cm (0.787 in) to about 6 cm (2.36 in) long. In some embodiments, the strap has a length of up to 10.5 cm (4.13 in) such as 1, 1.5, 2.0, 2.5, 3.0, 3.5, 4.0, 4.5, 5.0, 5.5, 6.0, 6.5, 7, 7.5, 8, 8.5, 9, 9.5, 10, 10.5 cm or a length of less than or defined between any of the aforementioned length values. In some embodiments where the strap is formed of an elastic material, the length of the strap can increase during stretching. Depending on the elasticity of the material, the length of the strap can increase, for example from less than 1% to 100% or it can increase from more than 1 to 500% or more, including, for example, 1-2, 2-3, 3-5, 5-8,8-10, 10-15, 15-20, 20-30, 30-40, 40-50, 50-60, 60-70, 70-80, 80-90, 90-95, 95-99, 99-100, 100-150, 150-200, 200-300, 300-400, 400-500 or more when the finger inserted into the loop. In some embodiments, the strap has a length of between of between 1 cm (0.394 in) and 10 cm (3.94 in) such as 1-10, 2-8, 3-8, 4-8, 4-7, or 5-6 cm when stretched in use on the proximal end of the plunger. The “in use” values listed above denote the length of the strap when the finger is inserted into the loop, not when the finger is aspirating the syringe (which, in some embodiments, can further increase the length of the strap).

The strap can be of any width suitable for providing support for the thumb or finger during syringe use. In some embodiments, the strap is between about 0.5 cm (0.197 in) to about 4 cm (1.57 in) wide. In some embodiments, the strap has a width of about 0.5, 1, 1.5, 2, 2.5, 3 cm or any amount on either side of or defined between the aforementioned length values. In some embodiments, where the strap is formed of an elastic material, the width of the strap can increase or decrease during stretching. In some embodiments, the strap can have varying width. For example, a portion of the strap can be about 1 cm (0.394 in) wide, and another portion of the cushion can be about 1.5 cm (0.591 in) wide.

In some embodiments, the strap can be of any thickness suitable for providing a comfortable surface for the thumb or finger during aspiration, for keeping the proximal end of the plunger close to the user's finger or thumb, or for both purposes. In some embodiments, the strap is between about 0.1 mm (0.00394 in) to about 10 mm (0.394 in) thick. In some embodiments, the cushion has a thickness of about 0.1, 0.5, 1, 1.5, 2, 2.5, 3, 3.5, 4, 4.5, 5, 5.5, 6, 6.5, 7, 7.5, 8, 8.5, 9, 9.5, 10 mm or any amount on either side of or defined between any one of the aforementioned thickness values. A strap can have varying thicknesses. For example, a portion of the strap can be about 1 mm (0.0394 in) thick and another portion of the cushion can be about 3 cm (1.181 in) thick. In some embodiments where the strap is formed of an elastic material, the thickness of the strap can decrease during stretching.

In some embodiments, a method of making a syringe involving a thumb grip is provided. In some embodiments, the method involves a strap (any one of the herein described straps) having a hole at both ends of the strap. The distal end of a plunger portion of the syringe can be inserted through each hole and the strap slid up to the proximal end of the plunger portion, where it can be restrained from falling off of the plunger. This restraint can be achieved by the plunger portion itself. For example, the proximal end (or the plunger tip 140) can be larger than the holes in the strap, thereby preventing the strap from sliding off of the plunger. The plunger can then be inserted in to a proximal portion of a barrel portion of a syringe. The insertion of the plunger portion through both holes in the strap can result in the formation of a loop around the proximal end of the plunger portion, thereby forming a loop for insertion of a user's thumb or finger.

As will be appreciated by one of skill in the art, when holes (which includes slits or types of openings) are used to attach the thumb grip to the plunger, the diameter of at least one of the holes should be smaller than the diameter of widest section of the proximal end of the plunger portion or the plunger tip, thereby allowing the widest section to prevent the thumb grip from coming off the syringe.

In some embodiments, a cushion is located on or within the thumb grip to make use more comfortable. In some embodiments, the cushion is configured to provide a comfortable surface while pushing down on the plunger portion. In some embodiments, the cushion is configured to provide a comfortable surface when the thumb grip is used to pull back on the plunger portion. In some embodiments, the thumb grip is used to pull the plunger portion proximally to aspirate. In other embodiments, the thumb grip is used to push the plunger portion distally to inject. In other embodiments, the thumb grip is used to both push the plunger portion distally to inject and alternately to pull the plunger proximally to aspirate.

In some embodiments, a method of using a thumb grip is provided. In some embodiments the method comprises providing a syringe comprising a barrel portion and a plunger portion comprising a distal end adapted for insertion into a proximal end of the barrel portion and providing a strap that is looped around a proximal end of the plunger portion. In some embodiments, the strap comprises, consists, or consists essentially of a first hole and a second hole. In some embodiments at least a part of the plunger portion is passed through the first hole and second hole. The strap is sized (either when stretched or not) to allow a finger or thumb to be inserted between the proximal end of the plunger portion and the strap. The strap can comprise a flexible, elastic, or flexible and elastic material. One can then insert a finger or thumb between the proximal end of the plunger and the strap and aspirate the syringe by exerting a force against the strap via the finger or thumb. In some embodiments, when the finger or thumb is inserted between the proximal end of the plunger and the strap, the strap is stretched. In some embodiments, the stretched strap exerts a constricting force on the finger or thumb.

In some embodiments the syringe includes a flange 40 on the proximal portion of the barrel portion of the syringe 10. The flange can be configured to be grasped by two adjacent fingers of the same hand. In some embodiments, the flange is circular. In some embodiments, the flange has two portions, each portion on opposite sides of the barrel portion. In some embodiments the flange 40 is rigid. In some embodiments, the flange can be grasped by two adjacent fingers of the same hand. In some embodiments, the flange is rotatable around the barrel portion. The flange can be integral to the barrel portion. In some embodiments, the flange is detachable from the barrel portion. The flange can completely encircle the barrel portion in some embodiments. In other embodiments, the flange partially encircles the barrel portion. In some embodiments, the flange is configured to be clipped onto the barrel portion.

The flange can extend about 1 mm (0.0394 in) to about 5 cm (1.97 in) or more out from the barrel portion. In some embodiments, the flange extends about 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15 mm, or any amount on either side of or defined between the aforementioned values out from the barrel portion. The flange can be of varying thickness, and is preferably of a comfortable thickness when grasped by two adjacent fingers of the same hand. In some embodiments, the flange has a thickness of about 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15 mm, or any amount on either side of or defined between the aforementioned thickness values.

In some embodiments, the flange includes finger loops to secure the fingers on the flange during pulling and pushing of the plunger portion. A finger loop can be rigid or flexible. The finger loop can be closed loops or in an open configuration. In some embodiments, the finger loop is a broken ring. The finger loop can be made of a variety of materials, including without limitation, rubber, cloth, plastic, elastic, metal, glass, neoprene, Gore-Tex®, silicone, Thermoplastic Elastomer (TPE), or any combination thereof.

In some embodiments, a cushion is located on the flange or finger loops to make use more comfortable. In some embodiments, a cushion is located adjacent to the flange and configured to provide cushioning when a force is exerted against the flange.

The flange can be rigid or flexible. In some embodiments, the flange is rigid. In some embodiments, the flange is rotatable. The flange can be made of a variety of materials, including without limitation, rubber, cloth, plastic, elastic, metal, glass, silicone, neoprene, Gore-Tex®, TPE, or any combination thereof. In some embodiments, the flange is made of rubber and has a textured surface.

The flange can be configured for single use or multiple use. In some embodiments, the flange is integral to the syringe. In some embodiments, the flange is detachable from the syringe. The flange can be attached to the syringe by a variety of means, including, for example without limitation Velcro® fastener, snaps, stretch fit, clamps, glue, tape, rubber cement, hooks, adhesives, magnet, mating grooves, interlocking parts, friction, suction, pins, ties, compression fit. In some embodiments, the flange is machined to closely fit the syringe. The thumb grip can be sterilized. A syringe can include more than one flange, located on either or both of the barrel portion or the plunger portion or cushions associated therewith.

As will be appreciated by one of skill in the art, when a cushion is associated or adjacent to a particular syringe structure, there can be embodiments in which there are additional intervening structures between the cushion and the syringe. For example, additional layers of padding, plastic, tape, attachment means, coatings, epoxy or glue, or other structures can be placed between the cushion and the syringe without preventing the cushion from being “associated with” or “adjacent to” the syringe or barrel portion. In embodiments in which there is necessarily nothing between the syringe or barrel portion and the cushion, the cushion can be described as being immediately adjacent, directly associated, or similar phrase.

In some embodiments, various aspects can be included together as a kit. In some embodiments, the kit includes at least two syringes. Each syringe can include the following: a barrel portion, a cushion positioned against the barrel portion (the cushion is configured to provide cushioning when the barrel portion is operably grasped by two adjacent fingers of a same hand), a plunger portion having a distal end adapted for insertion into a proximal end of the barrel portion, and a specified amount of a dermal filler contained within the barrel portion. In some embodiments, the barrel portion contains about 1 ml (0.034 oz.) or less of dermal filler. In some embodiments, 3, 4, 5, 6, 7, 8, 9, 10, or more syringes are packaged together.

In some embodiments the kit includes at least one syringe. The syringe has a barrel portion and a plunger portion. The plunger portion has a distal end adapted for insertion into a proximal end of the barrel portion. The kit also has a cushion configured to be positioned against the barrel portion. The cushion can provide cushioning when the barrel portion is operably grasped by two adjacent fingers of one hand. In some embodiments there is a specified amount of a dermal filler contained within the barrel portion. In some embodiments, the cushion is integral to the barrel portion of the syringe. In some embodiments, the cushion is detachable from the syringe. In other embodiments, the cushion is configured to be worn by the syringe operator. In some embodiments, the cushion is configured to be attached to a finger of the syringe operator, and a second cushion is configured to be attached to an adjacent finger of the same hand. In some embodiments, the cushion has an adhesive layer on the side to be positioned against the syringe, allowing one to apply the cushion to the syringe. In some embodiments the cushion is a part of a glove. In some embodiments the plunger is packaged separate from the barrel to be inserted during use. In some embodiments the plunger, barrel, are packaged pre-assembled.

In some embodiments, the kit or syringe is further associated with a needle. In some embodiments the needle is already attached to the syringe. In some embodiments, the needle is configured for the administration of a dermal filler. In some embodiments, the needle is configured for the administration of a dermal filler to the facial region of a subject. In some embodiments, the needle is smaller than a 23-gauge needle, for example 23-25, 25-27, 27, 27-28, 28-31, 28-29, 29-30, 30, 30-31 or smaller. In some embodiments, the needle is a 30-guage sized needle. In some embodiments, the needle is between 0.5 to 1.5 inches in length.

In some embodiments the kit includes at least one syringe and at least one strap (such as any strap described herein). In some embodiments the strap has a first hole at a first end and a second hole at a second end of the strap. In some embodiments, the kit further includes a cushion positioned against the barrel portion.

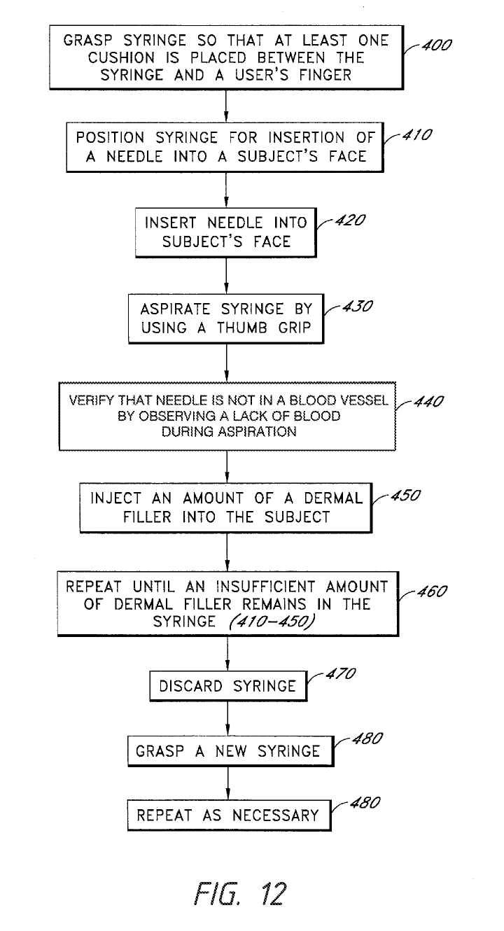

In some embodiments, a method of comfortably administering a liquid can be achieved by using one or some of the above embodiments. FIG. 12 depicts an embodiment of a method of using some embodiments of an ergonomic syringe. Not all of the steps need be included and not all of the below described steps have been described in the figure.

In some embodiments, a user can grip a syringe 10, (e.g. as disclosed in FIG. 1-FIG. 3) so that at least a portion of the cushion 50, 90, 150 is positioned between the user's fingers and the syringe 10 (as shown in step 400). In some embodiments, the user grips the syringe 10 by holding, gripping, or pressing onto or against the cushion. In some embodiments, the user holds the syringe via the inner sides of two adjacent fingers. In some embodiments, the user holds the syringe using primarily the index finger and the middle finger. In some embodiments, the user holds the syringe in a single hand.

The user can then manipulate the syringe for placement of the syringe (step 410) and insertion of the needle into the subject (step 420). In some embodiments, the user uses a single hand to do one or both of these operations. In some embodiments, the user uses two fingers to position and insert the needle of the syringe into the subject. In some embodiments the syringe is gripped by the index and middle fingers during the insertion of the needle into the subject. In some embodiments the needle is inserted into a location that will benefit from the injection of a dermal filler. In some embodiments, the injections are made to the face of a subject.

In some embodiments, the user can then optionally aspirate the syringe to verify that the needle has not been inserted into a portion of the body, into which injection of the liquid is not desired (e.g. for injection of a dermal filler, a vein or artery (step 430)). In some embodiments, this is done by withdrawing the plunger portion slightly and observing if blood is taken into the syringe or needle (step 440). In some embodiments, this is achieved by using the thumb of the hand holding the syringe and withdrawing the plunger via the thumb grip. If too much blood is observed during aspiration (to indicate that the needle was inserted into a blood vessel), the needle can be withdrawn from the subject and inserted elsewhere.

In some embodiments, aspiration is simplified by including a flange, thumb hook, or thumb grip on the plunger. In some embodiments, the thumb grip is a loop (opened or closed). In some embodiments, the thumb grip has a cushion associated with it. Thus, in some embodiments, while one operates the thumb grip, a cushion is located between at least a portion of the user's hand (which includes fingers) and a remaining part of the syringe. Thus, in an embodiment where one uses a plunger portion 30 as shown in FIG. 4, the padding 170 is placed between the location where the thumb is to be positioned and the rest of the thumb grip

In some embodiments, the cushion is the entire thumb grip itself, thus, aspiration can involve simply manipulating the cushion, for example, by moving the cushion (or strap) away from the syringe body. Such a method would include embodiments involving the use of the plunger portions depicted in FIG. 5, or the optional hole shown in FIG. 1. As will be appreciated by one of skill in the art, in some embodiments, a finger can be used to operate the thumb grip instead of a thumb. In some embodiments while one operates the thumb grip, the thumb grip may actually be against the heel or palmar aspect of the operator's hand.

In some embodiments, the user can then depress the plunger portion to inject the flowable substance into the subject (step 450). In some embodiments, the user at least grips the syringe by the inside portion of two adjacent fingers on one hand while depressing the plunger portion. In some embodiments, at least a portion of at least one cushion will be located between at least one of the user's fingers and the body of the syringe or the plunger of the syringe. By compressing the syringe, the user will transfer stress or force to the cushion, which can alter the distribution of the forces felt by the user so that a more comfortable and/or secure feeling grip will result. In some embodiments, the plunger is depressed by the user's thumb. In some embodiments, the plunger is depressed by another part of the user's hand, such as the palm of the hand.

In some embodiments, the user will inject a substance related to cosmetics. For example, the user can inject a dermal filler. In some embodiments, the substance is a dermal filler and the placement of the injection is determined by where an amount of dermal filler should be injected. In some embodiments the user can use a dermal filler such as Juvederm®, Juvederm XC®, and or their “Plus” products, and or, Volluma®, Vollure®, or any Hyaluronic Acid dermal filler. The dermal filler may be a collagen type filler, or a particulate type filler like Radiance® or Aquamid®, or Isologen®. In certain embodiments a substance such as a numbing agent is injected. Such substances include but are not limited to: Lidocaine® or Carbocaine®, or the like, for certain surgeries or dental procedures

In some embodiments, the substance is PERLANE® hyaluronic acid derivative dermal filler. In some embodiments, less than 2 ml (0.068 fl. oz.) of the substance is injected, for example 2-1.5, 1.5-1, 1-0.5, 0.5-0.4, 0.4-0.3, 0.3-0.2, 0.2-0.1, 0.1-0.05, 0.05-0.01 ml, or less. As will be appreciated by one of skill in the art, the method need not be limited to cosmetic or medical issues as any application of a syringe could benefit from certain embodiments of the described invention. For example, in some embodiments, the substance is glue and is injected into cracks in wood that are to be repaired.

In some embodiments, the user repeats the above steps (e.g., steps 410-450) multiple times with a single syringe (e.g., step 460). In some embodiments, the user repeats the above steps 3 to 20 times with a single syringe. In some embodiments a single syringe can be used to make 3-8 injections (and optional aspirations) in a face of a subject. In some embodiments, the user uses multiple syringes in a single treatment session. For example, the user can use between 2 and 50 syringes in any one session. In some embodiments, the user disposes of each syringe when the syringe is empty (step 470). In some embodiments, the user then grasps a new syringe that is prefilled with the substance (step 401). In some embodiments the substance is a dermal filler. In some embodiments, approximately 0.1 ml (0034 fl. oz.) of the substance is injected with each proper insertion of the needle and approximately 5-10 pre-filled syringes are used on a single subject in one sitting. As will be appreciated by one of skill in the art, the above steps can be repeated as necessary in order to achieve the desired goal (step 480).

In some embodiments, the use of the thumb grip allows for single-handed aspiration in order to determine if the needle is in a vessel. In some embodiments, the use of the thumb grip allows for immediate injection, post aspiration, without moving and/or losing the syringe and/or needle placement. In some embodiments, the use of the disclosed device can result in an improvement in the aspiration/injection approximation, as two-handed aspiration can result in significant displacement of the needles. Thus, in some embodiments, use of the device as described herein allows for a method having some or all of the above described advantages.

In some embodiments, the user can apply the cushions to the hands or fingers, rather than using cushions that are fixed to the syringe. For example, the cushions can be placed on the user's fingers, or gloves, or be a part of the gloves, especially the index and middle fingers and especially on the inside of these two fingers. Similarly, a cushion can be placed on the user's thumb. The cushion can include substances such as band-aids, tape, foam tape, adhesives, gels, etc. Preferably, the cushion does not interfere with the use of gloves.

The various devices and systems described above provide a number of ways to carry out the invention. It is to be understood that not necessarily all objectives or advantages described can be achieved in accordance with any particular embodiment described herein. Also, although the invention has been disclosed in the context of certain embodiments and examples, it will be understood by those skilled in the art that the invention extends beyond the specifically disclosed embodiments to other alternative embodiments and/or uses and obvious modifications and equivalents thereof. Accordingly, the invention is not intended to be limited by the specific disclosures of preferred embodiments herein. Unless denoted otherwise in the above specification by an explicitly recited exclusion, embodiments described as “including,” “having,” “comprising,” etc., are also contemplated as consisting and consisting essentially of the recited elements. This only applies to the use of these terms in the specification and does not apply to the use of the terms in the claims.

While various embodiments of the present invention have been described in detail, it is apparent that modifications and alterations of those embodiments will occur to those skilled in the art. However, it is to be expressly understood that such modifications and alterations are within the scope and spirit of the present invention. Further, the inventions described herein are capable of other embodiments and of being practiced or of being carried out in various ways. In addition, it is to be understood that the phraseology and terminology used herein is for the purposes of description and should not be regarded as limiting. The use of “including,” “comprising,” or “adding” and variations thereof herein are meant to encompass the items listed thereafter and equivalents thereof, as well as, additional items.

What is claimed is:

1. A system for providing injections comprising:
a gripping device comprising an open loop structure having a first end and a second end;
the gripping device further comprising an attachment slot structure, the attachment slot structure comprising a recess;
a first finger flange having an attachment feature; and
a second finger flange having an attachment feature,
wherein the attachment slot structure is configured to attach to a proximal end of a plunger portion of a syringe,
wherein the recess is configured to receive a shaft of the plunger portion of the syringe,
wherein the attachment feature of the first finger flange is configured to attach to a proximal portion of a barrel portion of the syringe, and
wherein the attachment feature of the second finger flange is configured to attach to the barrel portion of the syringe, distally located from the first finger flange.

2. The system of claim 1 wherein, the second finger flange is configured to be attached to the barrel portion of the syringe wherein the second finger flange is rotatively and slidable adjustable in relation to the barrel portion of the syringe.

3. The system of claim 1, wherein the first end of the open loop structure extends radially from the second between more than 180-degrees, but less than 315-degrees from the second end of the open loop structure.

4. The system of claim 3, wherein the attachment slot structure further comprises a first retention feature;
wherein the first retention feature is configured to retain the proximal end of the plunger portion within the attachment slot structure when the proximal end of the plunger portion is disposed within the attachment slot structure.

5. The system of claim 4, wherein the attachment slot structure further comprises a second retention feature.

6. The system of claim 4, wherein the first retention feature comprises a wedge aspect which protrudes from a surface of the slot structure,
wherein the wedge aspect is configured to provide resistance against the removal of the proximal end of the plunger portion from the attachment slot structure.

7. The system of claim 4, wherein the recess comprises a keyhole shape.

8. The system of claim 7, wherein the recess further comprises a first knob configured to restrict movement between the proximal end of the plunger portion of the syringe and the gripping device.

9. The system of claim 4, wherein the first end further comprises an enlarged portion.

10. The system of claim 1, wherein the second finger flange comprises an engagement feature and a collar portion, the collar portion having an arc angle in excess of 180-degrees.

11. The system of claim 10, wherein engagement feature comprises a tapered form and the collar portion is configured to affixably mate with the barrel portion of the syringe.

12. The system of claim 11, wherein the interface between the engagement feature and the collar portion results in a first snap-point and a second snap-point, the distance between the first snap-point and the second snap-point being less than the width of the barrel portion of the syringe.

13. The system of claim 12, wherein the second finger flange further comprises a rub structure consistent with a first surface of the second finger flange.

14. The system of claim 13, wherein the rib structure is consistent with the collar portion of the second finger flange.
